(19) United States
(12) Patent Application Publication
(10) Pub. No.: US 2010/0130862 A1
(43) Pub. Date: May 27, 2010

(54) Providing Volume Information On A Periodically Moving Target Object In An Ultrasound System

(76) Inventor: Jae Heung Yoo, Seoul (KR)

Correspondence Address: JONES DAY, 222 EAST 41ST ST, NEW YORK, NY, 10017, US

(21) Appl. No.: 12/625,311

(22) Filed: Nov. 24, 2009

(30) Foreign Application Priority Data
Nov. 25, 2008 (KR) 10-2008-0117313
Nov. 2, 2009 (KR) 10-2009-0104738

(51) Int. Cl. A61B 8/14

(52) U.S. Cl. 600/443

(57) ABSTRACT

Embodiments for providing volume information of a periodically moving target object with an ultrasound system are disclosed. An ultrasound data acquisition unit is configured to transmit/receive ultrasound signals from/to a periodically moving target object to thereby form ultrasound data. A processing unit is coupled to the ultrasound data acquisition unit. The processing unit is configured to form volume data including a plurality of frames based on the ultrasound data, set a moving period of the target object based on the volume data, reconstruct the volume data into a plurality of sub-volume data based on the moving period and measure volume of the target object based on the plurality of sub-volume data to thereby form volume information.

The present application claims priority from Korean Patent Applications Nos. 10-2008-0117313 and 10-2009-0104738 filed on Nov. 25, 2008 and Nov. 2, 2009, respectively, the entire subject matters of which are incorporated herein by reference.

### TECHNICAL FIELD

The present disclosure relates to ultrasound systems, and more particularly to providing volume information regarding a periodically moving target object in an ultrasound system.

### BACKGROUND

An ultrasound system has become an important and popular diagnostic tool since it has a wide range of applications. Specifically, due to its non-invasive and non-destructive nature, the ultrasound system has been extensively used in the medical profession. Modern high-performance ultrasound systems and techniques are commonly used to produce two or three-dimensional diagnostic images of internal features of an object (e.g., human organs).

Recently, the ultrasound system has been improved to provide a three-dimensional ultrasound image. A static three-dimensional ultrasound image is often used for ultrasound diagnostic purposes. By using the static three-dimensional ultrasound image, it is possible to perform accurate observations, diagnoses or treatments of a human body without conducting complicated procedures such as invasive operations. However, the static three-dimensional image may not be useful in certain cases, for example, in observing a moving target object such as a heart, a fetus in the uterus or the like in real time.

Further, there has been an increased interest in heart conditions of a fetus since there is an increasing need to perform an early diagnosis of the fetus' status. However, it is impossible to measure exact volume of the moving heart.

### SUMMARY

Embodiments for forming an elastic image in an ultrasound system are disclosed herein. In one embodiment, by way of non-limiting example, an ultrasound system comprises: an ultrasound data acquisition unit configured to transmit/receive ultrasound signals from/to a periodically moving target object to thereby form ultrasound data; and a processing unit coupled to the ultrasound data acquisition unit, the processing unit being configured to form volume data including a plurality of frames based on the ultrasound data, to set a moving period of the target object based on the volume data, to reconstruct the volume data into a plurality of sub-volume data based on the moving period and to measure volume of the target object based on the plurality of sub-volume data to thereby form volume information.

In another embodiment, a method of providing volume information of a periodically moving target object, comprises: a) transmitting/receiving ultrasound signals from/to a periodically moving target object to thereby form ultrasound data; b) forming volume data including a plurality of frames based on the ultrasound data; c) setting a moving period of the target object based on the volume data; d) reconstructing the volume data into a plurality of sub-volume data based on the moving period; e) setting contour of the target object on each of the sub-volume data; and f) measuring volume of the target object based on the contour to thereby form volume information.

In yet another embodiment, a computer readable medium comprising computer executable instructions is configured to perform the following acts: a) transmitting/receiving ultrasound signals from/to a periodically moving target object to thereby form ultrasound data; b) forming volume data including a plurality of frames based on the ultrasound data; c) setting a moving period of the target object based on the volume data; d) reconstructing the volume data into a plurality of sub-volume data based on the moving period; e) setting contour of the target object on each of the sub-volume data; and f) measuring volume of the target object based on the contour to thereby form volume information.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a block diagram showing an illustrative embodiment of an ultrasound data acquisition unit.

FIG. 3 is a block diagram showing an illustrative embodiment of a processing unit.

FIG. 4 is a block diagram showing an illustrative embodiment of a period detecting section.

FIG. 5 is a flowchart showing an illustrative embodiment of forming volume information of a periodically moving target object.

FIG. 6 is a schematic diagram showing an example of acquiring ultrasound data corresponding to a plurality of frames.

FIG. 7 is a schematic diagram showing an example of setting a feature point at each of the slice images.

FIG. 8 is a schematic diagram showing an example of forming a feature point curve based on distances between a principal axis and feature points.

FIG. 9 is a schematic diagram showing an example of a feature point curve.

FIG. 10 is a schematic diagram showing a procedure of reconstructing volume data based on a moving period of a target object.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

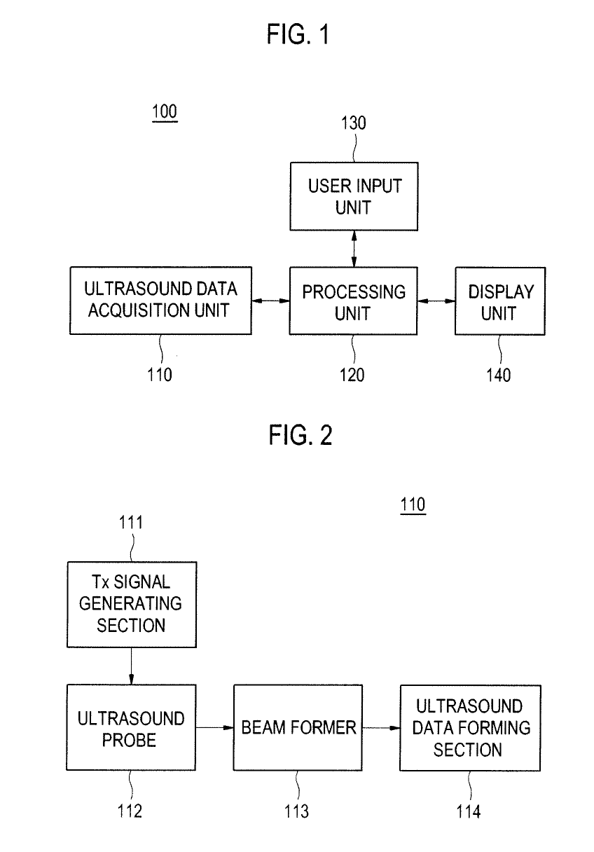

Referring to FIG. 1, an ultrasound system 100 in accordance with an illustrative embodiment is shown. As depicted therein, the ultrasound system 100 may include an ultrasound data acquisition unit 110. The ultrasound data acquisition unit 110 may be operable to transmit/receive ultrasound signals to/from a periodically moving target object to thereby output ultrasound data. The ultrasound data acquisition unit 110 may include a transmit (Tx) signal generating section 111, as shown in FIG. 2.

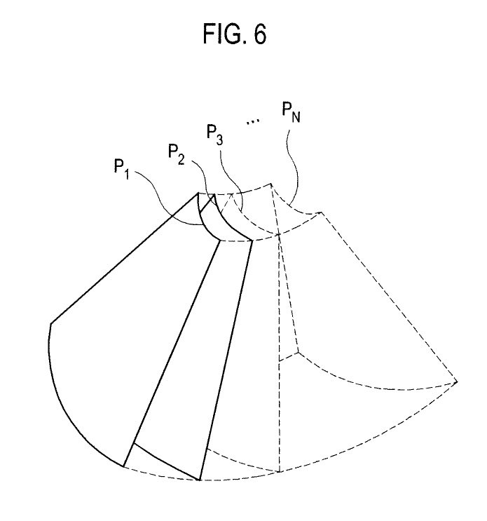

The Tx signal generating section 111 may be operable to generate Tx signals. The Tx signal generating section 111 may perform the generation of the Tx signals at every predetermined time to thereby form a plurality of Tx signals for obtaining each of frames Pᵢ(1≦i≦N) representing the target object, as shown in FIG. 6. The frame may represent sectional planes of the target object.

The ultrasound data acquisition unit 110 may further include an ultrasound probe 112 containing a plurality of elements for reciprocally converting ultrasound signals and electrical signals. The ultrasound probe 112 may be operable to transmit ultrasound signals into the target object in response to the Tx signals. The ultrasound probe 112 may be further operable to receive echo signals reflected from the target object to thereby output received signals. The received signals may be analog signals. The ultrasound probe 112 may include a three-dimensional probe, a two-dimensional array probe or the like.

The ultrasound data acquisition unit 110 may further include a beam former 113. The beam former 113 may be operable to convert the received signals into digital signals. The beam former 113 may be further operable to apply delays to the digital signals in consideration of distances between the elements and focal points to thereby output digital receive-focused signals.

The ultrasound data acquisition unit 110 may further include an ultrasound data forming section 114. The ultrasound data forming section 114 may be operable to form ultrasound data based on the digital receive-focused signals.

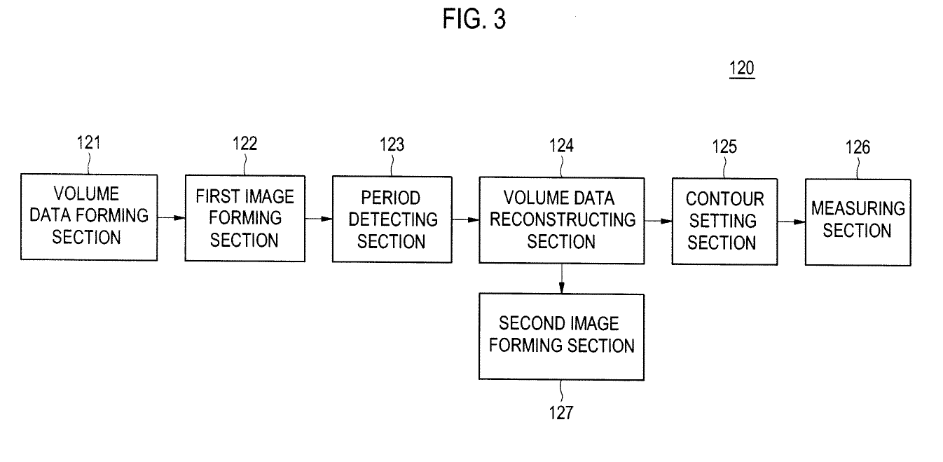

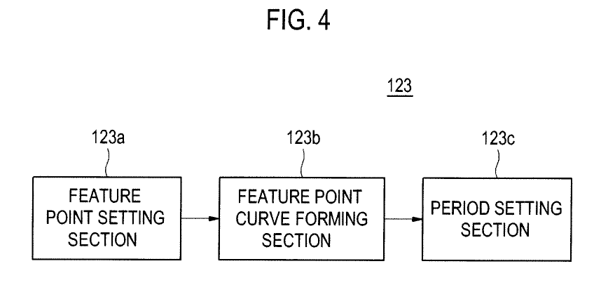

Referring back to FIG. 1, the ultrasound system 100 may further include a processing unit 120, which may be coupled to the ultrasound data acquisition unit 110. Referring to FIG. 3, the processing unit 120 may include a volume data forming section 121, a first image forming section 122, a period detecting section 123, a volume data reconstruction section 124, a contour setting section 125, a measuring section 126 and a second image forming section 127. Also, the period detecting section 123 may include a feature point setting section 123a, a feature point curve forming section 123b and a period setting section 123c, as shown in FIG. 4.

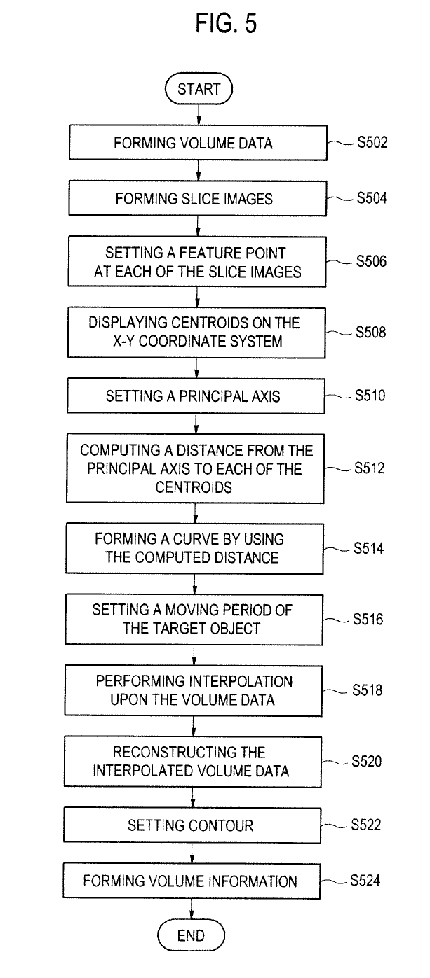

FIG. 5 is a flowchart showing an illustrative embodiment of forming volume information of a periodically moving target object. Referring to FIG. 5, it will be explained as to how the volume information is formed with respect to the periodically moving target object.

The volume data forming section 121 may be operable to synthesize the ultrasound data corresponding to the frames Pᵢ(1≦i≦N) to thereby form volume data including the frames Pᵢ(1≦i≦N), as shown in FIG. 5 (S502).

The first image forming section 122 may be operable to form the plurality of slice images based on the volume data (S504). The slice image may be a brightness mode image corresponding to the frame. Also, the slice image may include pixels each having a brightness value.

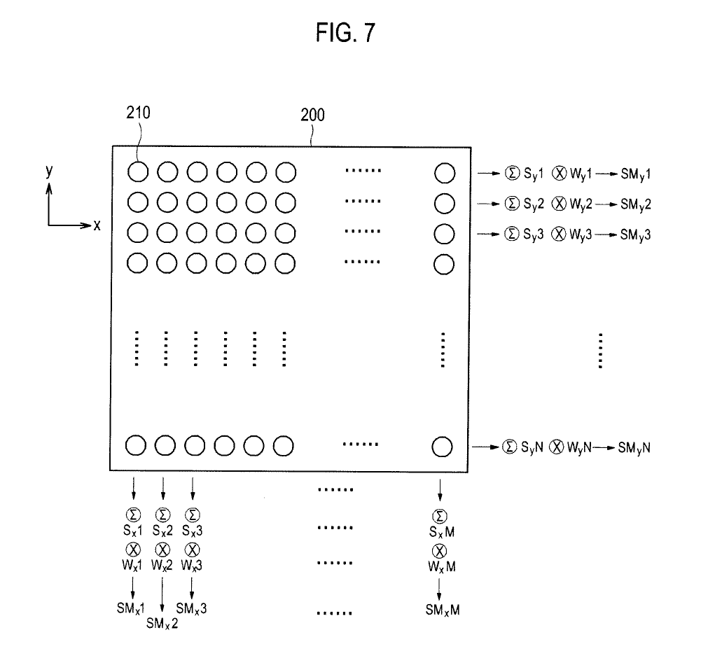

The feature point setting section 123a may be operable to set a feature point on each of the slice images formed by the first image forming section 122 (S506). The feature point may be set by using common feature on each of the slice images. In one embodiment, the feature point may be set by using a centroid of brightness values constituting each of the slice images. A method of determining a centroid of brightness values will be described by using a slice image 200 having M×N pixels 210, as shown in FIG. 7 as an example.

FIG. 7 is a schematic diagram showing an example of setting the feature point at each of the slice images. For the sake of convenience, it will be described that the slice images are placed on the X-Y plane of rectangular coordinate system in which the X coordinates of the slice image range from 1 to M and the Y coordinates of the slice image range from 1 to N. The feature point setting section 123a may be operable to vertically sum the pixel values at each of the X coordinates 1 to M in the slice image. That is, assuming that brightness values in the slice image are represented by P_{XY}, the feature point setting section 123a may be operable to sum P_{X1}, P_{X2}, . . . and P_{XN} to thereby output first sums Sx1-SxM corresponding to the respective X coordinates. Subsequently, the feature point setting section 123a may further multiply the first sums Sx1-SxM by weights Wx1-WxM, respectively, to thereby output first weighted sums SMx1-SMxM. In one embodiment, the weights Wx1-WxM may be determined by arbitrary values, which increase or decrease at a constant interval. For example, the numbers 1-M may be used as the weight values Wx1-WxM. The feature point setting section 123a may be further operable to sum all of the first sums Sx1-SxM to thereby output a second sum, as well as sum all of the first weighted sums SMx1-SMxM to thereby output a third sum. The feature point setting section 123a may further divide the third sum by the second sum. It may then set the division result as the centroid on the X axis.

Also, the feature point setting section 123a may be operable to horizontally sum the pixel values at each of the Y coordinates 1-N in the slice image. That is, assuming that brightness values in the slice image are represented by P_{XY}, the feature point setting section 123a may be operable to sum P_{1Y}, P_{2Y}, . . . and P_{MY} to thereby output fourth sums Sy1-SyN corresponding to the respective Y coordinates. Subsequently, the feature point setting section 123a may further multiply the fourth sums Sy1-SyN by weights Wy1-WyN, respectively, to thereby output second weighted sums SMy1-SMyN. In one embodiment, the weights Wy1-WyN may be determined by arbitrary values, which increase or decrease at a constant interval. For example, the numbers 1-N may be used as the weight values Wy1-WyN. The feature point setting section 123a may be further operable to sum all of the fourth sums Sy1-SyN to thereby output a fifth sum, as well as sum all of the second weighted sums SMy1-SMyN to thereby output a sixth sum. The feature point setting section 123a may further divide the sixth sum by the fifth sum and then set the division result as the centroid on the Y axis.

Although it is described that the feature point is set by using the centroid of brightness values constituting each of the slice images, the feature point setting is certainly not limited thereto. The feature point at each of the slice images may be set through singular value decomposition upon each of the slice images.

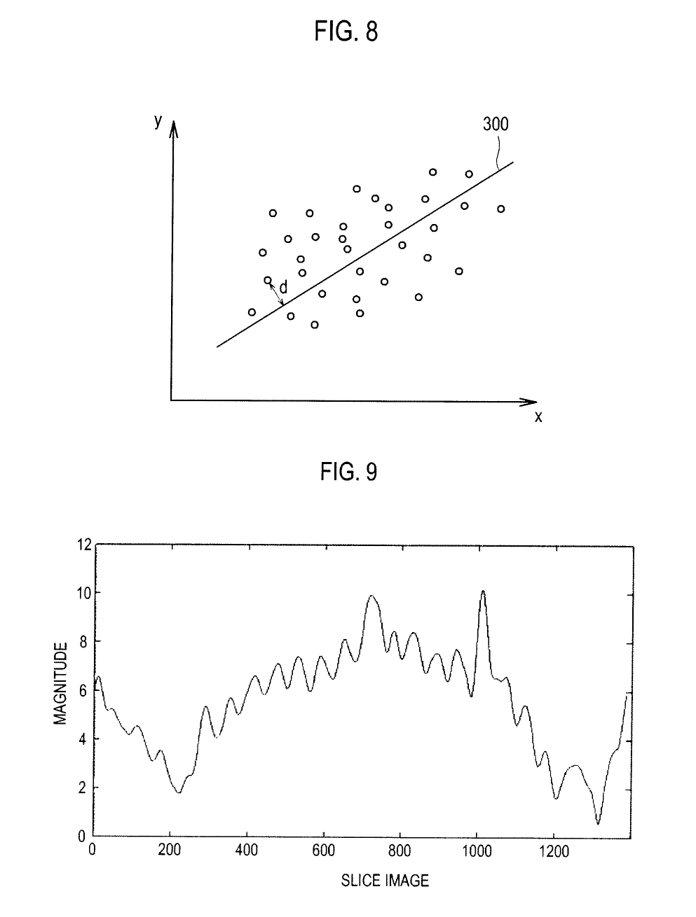

Once the setting of the centroid is complete for all of the slice images, the feature point curve forming section 123b may display centroids on the X-Y coordinate system (S508) and then set a principal axis 300 thereon (S510), as illustrated in FIG. 8.

FIG. 8 is a schematic diagram showing an example of forming the feature point curve based on distances between the principal axis and feature points. The feature point curve forming section 123b may be further operable to compute distances “d” from the principal axis 300 to each of the centroids (S512). The feature point curve forming section 123b may further form a curve by using the computed distances (S514), as illustrated in FIG. 9.

FIG. 9 is a schematic diagram showing an example of the feature point curve. The period setting section 123c may be operable to set a moving period of the target object by using peak points in the graph illustrated in FIG. 9 (S516). In one embodiment, the period setting section 123c may be operable to calculate the gradients in the curve in FIG. 9. The period setting section 123c may further calculate zero cross points, the gradient of which changes from positive to negative, and then detect the zero cross points having a similar distance, thereby setting a period of the detected zero cross points to the moving period of the target object.

Referring back to FIG. 5, the volume data reconstructing section 124 may be operable to interpolate the volume data to have the same number of the frames within each of the periods (S518). After completing the interpolation, the volume data reconstructing section 124 may further reconstruct the interpolated volume data (S520).

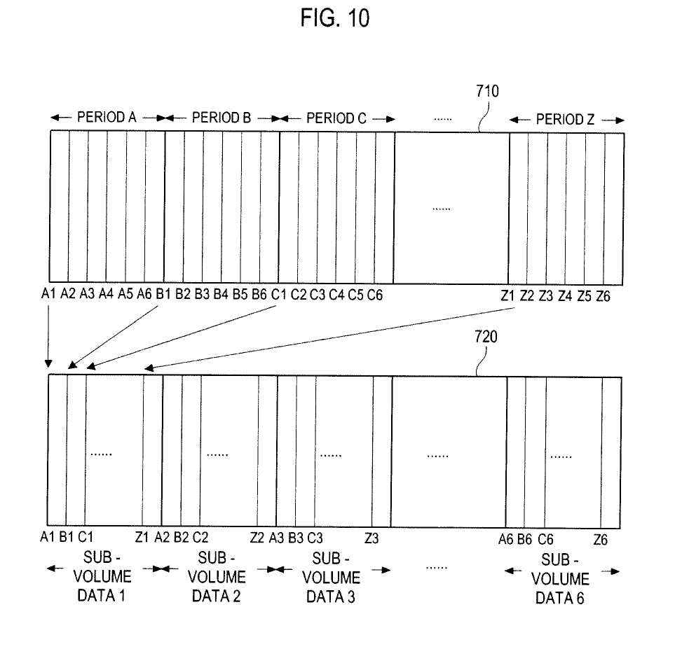

FIG. 10 shows a procedure of reconstructing the interpolated volume data. As shown in FIG. 10, twenty-six local periods A to Z exist in one volume data 710. Assuming that six frames are contained in each of periods in the interpolated volume data as shown in FIG. 10, the reconstructed volume data 720 may include six sub-volume data. Each of the sub-volume data may include 26 frames Aᵢ to Zᵢ.

Further, when the volume data are acquired by scanning the target object, the object (e.g., expectant mother or fetus) may be moved. This makes it difficult to accurately detect the heartbeat of the fetus. Accordingly, the volume data reconstructing section 124 may be further operable to compensate the motion of the expectant mother or the fetus by matching the brightness of pixels. The detailed description relating to the method of compensating the motion is omitted herein since the conventional methods may be used.

The contour setting section 125 may be operable to set contour of the target object on each of the sub-volume data (S522). In one embodiment, the contour setting section 125 may be operable to set the contour on each of the sub-volume data based on a user instruction from a user input unit 130. In another embodiment, the contour setting section 125 may automatically detect contour points at each of the sub-volume data to thereby set the contour on each of the sub-volume data based on the detected contour points. The detailed description relating to the method of detecting the contour points is omitted herein since the conventional methods may be used.

The measuring section 126 may measure volume of the target object based on the contour of the target object set by the contour setting section 125 to thereby form volume information (S524). The detailed description relating to the method of measuring the volume with the contour is omitted herein since the conventional methods may be used.

Referring back to FIG. 3, the second image forming section 127 may be operable to render each of the sub-volume data to thereby form three-dimensional ultrasound images. A user may input a user instruction to set a contour of the target object by using three-dimensional ultrasound images.

Referring again to FIG. 1, the ultrasound system 100 may further include the user input unit 130. The user input unit 130 may be operable to receive the user instruction from a user. The user instruction may include an instruction to set the contour of the target object on each of the three-dimensional ultrasound images. The user input unit 130 may include a control panel (not shown), a mouse (not shown), a keyboard (not shown) or the like.

The ultrasound system 100 may further include a display unit 140. The display unit 140 may display the volume information from the processing unit 120. The display unit 140 may further display the three-dimensional ultrasound images from the processing unit 120.

In another embodiment, the present invention may provide a computer readable medium comprising computer executable instructions configured to perform following acts: a) transmitting/receiving ultrasound signals from/to a periodically moving target object to thereby form ultrasound data; b) forming volume data including a plurality of frames based on the ultrasound data; c) setting a moving period of the target object based on the volume data; d) reconstructing the volume data into a plurality of sub-volume data based on the moving period; e) setting contour of the target object on each of the sub-volume data; and f) measuring volume of the target object based on the contour to thereby form volume information. The computer readable medium may comprise a floppy disk, a hard disk, a memory, a compact disk, a digital video disk, etc.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
an ultrasound data acquisition unit configured to transmit/receive ultrasound signals from/to a periodically moving target object to thereby form ultrasound data; and
a processing unit coupled to the ultrasound data acquisition unit, the processing unit being configured to form volume data including a plurality of frames based on the ultrasound data and set a moving period of the target object based on the volume data, the processing unit being further configured to reconstruct the volume data into a plurality of sub-volume data based on the moving period and measure volume of the target object based on the plurality of sub-volume data to thereby form volume information.

2. The ultrasound system of claim 1, wherein the processing unit comprises:
a volume data forming section configured to form the volume data based on the ultrasound data;
a first image forming section configured to form a plurality of slice images based on the volume data;
a period detecting section configured to set a feature point on each of the slice images and set the moving period of the target object based on the feature points set on the slice images;
a volume data reconstructing section configured to interpolate the volume data and reconstruct the interpolated volume data into the plurality of sub-volume data based on the moving period;
a contour setting section configured to set contour of the target object on each of the sub-volume data; and
a measuring section configured to measure the volume of the target object based on the contour to thereby form the volume information.

3. The ultrasound system of claim 2, wherein the period detecting section comprises:
a feature point setting section configured to set the feature point on each of the slice images based on brightness values of pixels included therein;
a feature point curve forming section configured to form a feature point curve based on the feature points; and
a period setting section configured to set the moving period of the target object based on the feature point curve.

4. The ultrasound system of claim 3, wherein the feature point setting section is configured to set a centroid of the brightness values on each of the slice images to the feature point.

5. The ultrasound system of claim 3, wherein the feature point curve forming section is configured to set a principal axis based on positions of the feature points and form the feature point curve based on distances between the feature points and the principal axis.

6. The ultrasound system of claim 3, wherein the period setting section is configured to calculate gradients from the feature point curve, detect zero crossing points that a sign of the gradient changes from positive to negative and determine the moving period based on intervals between the detected zero crossing points.

7. The ultrasound system of claim 2, wherein the processing unit further comprises a second image forming section configured to render the plurality of sub-volume data to thereby form three-dimensional ultrasound images.

8. The ultrasound system of claim 7, further comprising a user input unit configured to receive a user instruction for setting the contour of the target object on each of the three-dimensional ultrasound images.

9. The ultrasound system of claim 8, wherein the contour setting section is configured to set the contour of the target object on each of the sub-volume data based on the user instruction.

10. The ultrasound system of claim 2, wherein the contour setting section is configured to detect counter points at each of the sub-volume data and set the contour on each of the sub-volume data based on the detected contour points.

11. A method of providing volume information of a periodically moving target object, comprising:
a) transmitting/receiving ultrasound signals from/to a periodically moving target object to thereby form ultrasound data;
b) forming volume data including a plurality of frames based on the ultrasound data;
c) setting a moving period of the target object based on the volume data;
d) reconstructing the volume data into a plurality of sub-volume data based on the moving period;
e) setting contour of the target object on each of the sub-volume data; and
f) measuring volume of the target object based on the contour to thereby form volume information.

12. The method of claim 11, wherein the step c) comprises:
c1) forming a plurality of slice images based on the volume data;
c2) setting a feature point on each of the slice images; and
c3) setting the moving period of the target object based on the feature points set on the slice images.

13. The method of claim 12, wherein the step c2) comprises:
setting the feature point on each of the slice images based on brightness values of pixels included therein.

14. The method of claim 13, wherein a centroid of the brightness values at each of the slice images is set to the feature point.

15. The method of claim 12, wherein the step c3) comprises:
c31) forming a feature point curve based on the feature points; and
c32) setting the moving period of the target object based on the feature point curve.

16. The method of claim 15, wherein the step c31) comprises:
setting a principal axis based on positions of the feature points; and
forming the feature point curve based on distances between the feature points and the principal axis.

17. The method of claim 15, wherein the step c32) comprises:
calculating gradients from the feature point curve;
detecting zero crossing points that a sign of the gradient changes from positive to negative; and
determining the moving period based on intervals between the detected zero crossing points.

18. The method of claim 11, wherein the step e) comprises:
setting the contour of the target object on each of the sub-volume data based on a user instruction.

19. The method of claim 11, wherein the step e) comprises:
detecting contour points of the target object at each of the sub-volume data; and
setting the contour on each of the sub-volume data based on the detected contour points.

20. A computer readable medium comprising computer executable instructions configured to perform following acts:
a) transmitting/receiving ultrasound signals from/to a periodically moving target object to thereby form ultrasound data;
b) forming volume data including a plurality of frames based on the ultrasound data;
c) setting a moving period of the target object based on the volume data;
d) reconstructing the volume data into a plurality of sub-volume data based on the moving period;
e) setting contour of the target object on each of the sub-volume data; and
f) measuring volume of the target object based on the contour to thereby form volume information.
